(19) United States
(12) Patent Application Publication
(10) Pub. No.: US 2014/0275871 A1
(43) Pub. Date: Sep. 18, 2014

(54) WIRELESS OPTICAL COMMUNICATION BETWEEN NONINVASIVE PHYSIOLOGICAL SENSORS AND PATIENT MONITORS

(71) Applicant: CERCACOR LABORATORIES, INC., Irvine, CA (US)

(72) Inventors: Marcelo M. Lamego, Cupertino, CA (US); Massi Joe E. Kiani, Laguna Niguel, CA (US)

(73) Assignee: CERCACOR LABORATORIES, INC., Irvine, CA (US)

(21) Appl. No.: 14/206,779

(22) Filed: Mar. 12, 2014

Related U.S. Application Data
(60) Provisional application No. 61/785,197, filed on Mar. 14, 2013.

(51) Int. Cl. A61B 5/00; A61B 5/1455

(52) U.S. Cl. CPC A61B 5/0022; A61B 5/1455; A61B 5/14552
USPC 600/316; 600/322; 600/323; 600/328

(57) ABSTRACT

Embodiments of the disclosure include a noninvasive physiological patient sensor and a patient monitor capable of wireless communication with one another. An optical communication path can be used to provide the communication path between the noninvasive physiological patient sensor and the patient monitor. The path can be maintained by one or more light sources and detectors traditionally associated with noninvasive optical sensors or by one or more additional dedicated light sources and detectors.

### REFERENCE TO RELATED APPLICATIONS

The present application claims priority benefit from U.S. Provisional Application No. 61/785,197, filed Mar. 14, 2013, entitled “WIRELESS OPTICAL COMMUNICATION BETWEEN NONINVASIVE PHYSIOLOGICAL SENSORS AND PATIENT MONITORS,” the disclosure of which is incorporated herein by reference in its entirety.

### BACKGROUND

1. Field of the Disclosure

The present disclosure generally relates to patient monitoring systems and, more specifically, embodiments of the present disclosure relate to optical communication between noninvasive physiological sensors and patient monitors.

2. Description of the Related Art

Medical device manufacturers are continually improving the processing capabilities of patient monitors, specifically of patient monitors that process signals based on attenuation of light by patient tissue, such as oximeters, co-oximeters, pulse oximeters, or other patient monitoring devices (“patient monitor” or “monitor”). In general, such patient monitoring systems include one or more optical sensors that irradiate tissue of a patient and one or more photodetectors that detect the radiation after attenuation thereof by the tissue. The sensor communicates the detected signal to a patient monitor, where the patient monitor may remove noise and preprocesses the signal. Advanced signal processors then perform processing to determine measurements of blood constituents or other physiological parameters of the patient.

In some patient monitoring systems, the sensor and patient monitor communicate using a wire communication link that physically connects the sensor and patient monitor. The wire communication link allows the sensor to provide signals to the patient monitor, such as signals responsive to light attenuated by body tissue carrying pulsing blood. The one or more processors of the patient monitor process the signals (or preprocessed signals responsive to the signals from the sensor) to determine measurement values for one or more physiological characteristics of a monitored patient. These characteristics can relate, for example, to pulse rate, hydration, overall wellness, trending information and analysis, or the like. In other embodiments, temperature and data stored in memory may be communicated along the sensor wire in the sensor cabling. The data stored in the memory may include source identifying information, sensor life information, some or all of the software programming for the processor, or the like. In addition, the wire communication link allows the patient monitor to provide calibration or control information to the sensor, such as, for example, to control the intensity of the light source.

### SUMMARY

A wire communication link, such as a cable, can reduce the portability and compactness of a patient monitoring system. Moreover, a wire communication link can stress internal components of a sensor and a patient monitor if tension or torsion is applied to the wire communication link, such as through patient movement. For example, a twisting or pulling of the wire communication link may transfer mechanical stress to the wire connections between a flex circuit or PCB board, and the wire communication link. As a result, improved systems and methods for communicating between sensors and patient monitors are desired.

The present disclosure includes a physiological monitoring system in which noninvasive physiological sensors and patient monitors can communicate using optical or light communication, in addition to or alternatively to communicating via a wired or other wireless communication. As used herein, the terms “optical communication” and “light communication,” in addition to having their ordinary meanings, can refer to communication between devices via changes in electromagnetic radiation. For instance, the electromagnetic radiation can include visible light, infrared light, or ultraviolet light, in some implementations. The changes in electromagnetic radiation can include, for example, a modulation of the electromagnetic radiation.

In an embodiment, a sensor includes one or more light sources and one or more detectors. The sensor can receive an optically transmitted sensor activation command and, after receipt of the sensor activation command, communicate one or more drive signals to at least one light source configured to irradiate tissue of a patient. At least one detectors can receive the light from the light source after attenuation by body tissue and can output a signal responsive to the detected light. The sensor can encode the detector output signal and communicate another drive signal to the at least one light source to transmit the encoded detector output signal using modulated light. In an embodiment, the sensor can use the same light source to irradiate tissue as is used to transmit data using modulated light, and in other embodiments, one or more different lights sources may be used. The sensor can receive a confirmation of data transfer from a receiving device of the transmitted data.

In an embodiment, a monitor includes a processor and a display that can display one or more physiological parameter measurements. The processor can transmit a sensor activation command to a sensor. The processor further can receive the forgoing encoded detector output signal. In an embodiment, the processor can receive this signal from a monitor-side detector, which can detect the modulated light transmitted by the sensor, and determine one or more physiological parameter measurements based at least on the one or more received signals. Upon receipt of the encoded detector output signal without errors, the processor may transmit a confirmation of data transfer to the sensor.

For purposes of summarizing the disclosure, certain aspects, advantages and novel features of the disclosure have been described herein. Of course, it is to be understood that not necessarily all such aspects, advantages or features will be embodied in any particular embodiment of the disclosure.

### BRIEF DESCRIPTION OF THE DRAWINGS

The following drawings and the associated descriptions are provided to illustrate embodiments of the present disclosure and do not limit the scope of the claims.

FIG. 1 illustrates light communication (“LC”) between a patient monitor and a noninvasive physiological sensor in a patient monitoring system.

FIG. 2A illustrates a simplified block diagram of the patient monitor of FIG. 1.

FIG. 2B illustrates a simplified block diagram of the noninvasive physiological sensor of FIG. 1.

FIG. 3 illustrates a simplified sensor measurement process.

FIG. 4 illustrates a simplified instrument measurement process.

FIG. 5 illustrates LC between a patient monitor and a noninvasive physiological sensor in a patient monitoring system.

### DETAILED DESCRIPTION

The disclosure herein includes embodiments in which a patient monitor and a noninvasive optical sensor communicate over a wireless optical communication path. In some embodiments, the path can employ one or more light sources and one or more light detectors of the noninvasive optical sensor used for parameter signal measurements. In other embodiments, the path can employ one or more additional light sources and one or more additional detectors on the noninvasive optical sensor. In some embodiments, the path can employ a display of the patient monitor to transmit modulated light (for instance, by repeatedly turning on and off a backlight of the display) detectable by one or more detectors of the noninvasive optical sensor. In other embodiments, the path can employ one or more additional light sources of the patient monitor to transmit modulated light detectable by the noninvasive optical sensor. In some embodiments, the path can employ a camera of the patient monitor to detect light transmitted by the noninvasive optical sensor. In other embodiments, the path can employ one or more additional detectors of the patient monitor to detect light transmitted by the noninvasive optical sensor. In various embodiments, the communication may be encrypted, fail after certain errors or signal strength issues and prompt a user to connect a cable, perform error-checking known to an artisan from the disclosure herein, perform custom error-checking, combinations of the same, or the like.

FIG. 1 illustrates light communication (“LC”) between a patient monitor 110 and a noninvasive physiological sensor 120 in a patient monitoring system 100A, according to an embodiment of the disclosure. The monitor 110 can include a handheld housing including an integrated touch screen 112 and an integrated detector 114 capable of light, photo, or video capture. In an embodiment, the screen 112 can include a 5.6″ LED backlit LCD with 1280×800 pixel resolution with 262,144 colors and a viewing angle of 179 degrees, although an artisan will recognize from the disclosure herein a wide variety of usable display devices. The monitor 110 can communicate information, such as a sensor activation command or calibration and control information, using modulated light L₁ from the screen 112 to detectors 124 of the sensor 120.

The sensor 120 can be, for example, a clothespin-style reusable optical sensor, in some mechanical respects similar to those employed in standard pulse oximetry. The sensor 120 may include sensor features, such as those disclosed in U.S. Pat. Nos. 6,580,086 and 8,203,704, titled “Multi-stream Sensor For Noninvasive Measurement of Blood Constituents,” each of which is incorporated by reference herein in its entirety. Specifically, the sensor 120 can include a light source, such as emitters 122 capable of emitting light of a variety of wavelengths. The detectors 124 can detect the light after attenuation by a tissue of the patient, such as, for example a digit of the patient. An artisan will further recognize from the disclosure herein that other sensors for measuring physiological parameters from other body tissue can employ wireless optical communication as disclosed herein, including, for example, an ear sensor, an organ sensor, a forehead sensor, a reflectance sensor, disposable sensors, reposable sensors, or the like. One or more temperature sensors or one or more memory devices may also be incorporated into the sensor 120. The sensor 120 can communicate information, such as one or more sensor signals responsive to detected light attenuated by body tissue, using modulated light L₂ transmitted by one or more of the emitters 122 to the detector 114 of the monitor 110.

The monitor 110 and sensor 120 can communicate using various forms of modulated light. One or more of the amplitude (e.g., intensity), phase, frequency, polarization, or the like of transmitted light can be modulated by the transmitting device based on a signal, and a detector of the receiving device can detect the light and extract the signal. For instance, the monitor 110 can modulate the amplitude of transmitted light by varying a supply voltage or current to a light source (not shown) within or behind the screen 112 based on a signal that the monitor 110 desires to transmit. In turn, at least one of the detectors 124 of the sensor 120 can detect the modulated light transmitted by the monitor 110 and extract the signal from the detected light. In another example, the sensor 120 can modulate the amplitude of transmitted light by varying a supply voltage or current to one or more of the emitters 122 in accordance with a signal to be transmitted, and the detector 114 of the monitor 110 can detect the modulated light transmitted by the sensor 120 and extract the signal from the detected light. In yet another example, the sensor 120 can modulate the frequency of light transmitted in accordance with a signal to be transmitted by selectively activating one or more of the emitters 122 where one or more individual emitters may emit light of a different frequency from the other emitters, and the detector 114 of the monitor 110 can detect the modulated light transmitted by the sensor 120 and extract the signal from the detected light.

In general, a user of the patient monitoring system 100A can interact with the monitor 110 to obtain and control one or more physiological parameter readings by the sensor 120. Upon sending a sensor activation command from monitor 110, the user may apply the sensor 120 to a digit, and the sensor 120 can obtain noninvasive physiological parameter measurements. The sensor 120 can communicate the measurements to the monitor 110, and the monitor 110 can output or further process the received measurements.

In an embodiment, the monitor 110 can be a smart phone. The smart phone may include software such as an application configured to manage measurement data received from the sensor 120. The smart phone can receive and analyze data from the sensor 120, display the data, and otherwise utilize the data to empower the user to take control of his or her health. Moreover, the application functionality of the monitor 110 can include trend analysis, current measurement information, alarms associated with below threshold readings or reminders to take measurement data at certain times or cycles, display customization, iconic data such as hearts beating, color coordination, bar graphs, gas bars, charts, graphs, or the like, all usable by a caregiver or smart phone user to enable helpful and directed medical monitoring of specified physiological parameters. In addition, the monitor 110 may be further configured as the handheld processing device disclosed in U.S. patent application Ser. No. 13/308,461, titled “Handheld Processing Device Including Medical Applications for Minimally and Non Invasive Glucose Measurements,” which is incorporated by reference herein in its entirety.

Although examples may described with respect to the embodiment shown in FIG. 1, an artisan will recognize from the disclosure herein alternative or additional functionality, user interaction mechanisms, or the like in the patient monitoring system 100A. For example, the housing of the monitor 110 can be shaped to ergonomically fit a user's hand, include more or less input mechanisms including, for instance, a connectable or slide-out keyboard, a pointing device, speech recognition applications, or the like. Moreover, the sensor 120 may be capable of wired communication or other wireless communication (for example, Wi-Fi™ or Bluetooth™) with the monitor 110. Furthermore, the monitor 110 can, for instance, be a table-top device or mountable on a wall, in addition to or alternatively to being a handheld device. Additionally, although the sensor 120 is illustrated as performing LC without a digit of a patient placed in the sensor 120, the sensor 120 can be configured to perform LC with a digit placed in the sensor 120. This can be accomplished, for instance, by controlling the intensity of transmitted light, sensitivity to detected light, placement of one or more emitters or detectors, or shape of the sensor 120 so that the sensor 120 can perform LC through or around the digit of the patient.

FIG. 2A illustrates a simplified block diagram of the monitor 110 of FIG. 1, according to an embodiment of the disclosure. As shown in FIG. 2A, the monitor 110 includes a processor 202, a memory 204, a user interface 206, an input/output 208, a LC transmitter 210, and a LC receiver 212. The processor 202 can execute a number of processes, including medical processes, signal processing, and application processing. In some embodiments, the processing can include parameter measurement processing that can be the same as or similar to those found in patient monitors manufactured by Masimo Corporation. The processor 202 can control the operations of the monitor 110 and use the memory 204 to store and retrieve patient data, physiological parameters measurements, received signals, or the like. The user interface 206 can enable a user of the monitor 110, such as a medical service provider or patient, to enter patient data and receive results of processes or measurements performed by the monitor 110. The monitor 110 can receive input data and output data through the input/output 208. The input/output 208 can include a serial port or wireless connectivity component such as a Wi-Fi™ or Bluetooth™ compliant transceiver, or the like. The LC transmitter 210 can include a light source and light source driver capable of transmitting modulated light, such as light having modulated amplitude, phase, frequency, or polarization. In an embodiment, the LC transmitter 210 can include a LED or other light source that provides light to illuminate a display for the user interface 206. The LC receiver 212 can include a detector that detects modulated light. The LC receiver 212 can transmit a detected signal to the processor 202 so that the processor 202 can demodulate the detected signal to extract data and decode the extracted data.

FIG. 2B illustrates a simplified block diagram of the sensor 120 of FIG. 1, according to an embodiment of the disclosure. As shown in FIG. 2B, the sensor 120 includes a processor or controller 222, a memory 224, a light source 226, a photodetector 228, a battery 230, a light source driver 232, an optional LC light source 234, and an optional LC light detector 236. The processor 222 can execute a number of processes, including medical processes and signal processing. The processor 222 can control the operations of the sensor 120 and use the memory 224 to store and retrieve medical data, physiological parameters measurements, signals responsive to light attenuation by patient tissue, and sensor signals. The light source 226 can include one or more emitters, such as the emitters 122 of FIG. 1, that can individually or together emit light of a variety of wavelengths. In an embodiment, the light source 226 can transmit modulated light, such as light having modulated amplitude, phase, frequency, or polarization. The photodetector 228 can include one or more detectors, such as the detectors 114 of FIG. 1, that can detect the light after attenuation by a body tissue of the patient. In an embodiment, the photodetector 228 can detect modulated light and generate one or more signals responsive to the modulated light. The photodetector 228 can transmit the one or more generated signals to the processor 222 for processing or transmission. The battery 230 can provide a power source for one or more components of the sensor 120. The light source driver 232 can generates driving signals for powering the light source 226 to irradiate tissue of a patient or to transmit data using modulated light. The optional LC light source 234 and LC light detector 236 can include a light source and detector, respectively, for transmitting and receiving LC using, for instance, modulated light. The light source driver 232 can generate driving signals for powering the LC light source 234 to transmit data using light.

FIG. 3 illustrates a sensor measurement process 300, according to an embodiment of the disclosure. The process 300 illustrates an example mode of operation of the sensor 120 in the patient monitoring system 100A of FIG. 1 and may be implemented by the various components of the sensor 120 of FIG. 2B. For convenience, the process 300 is described in the context of the patient monitoring system 100A of FIG. 1 and the sensor 120 of FIG. 2B, but the process 300 may instead be implemented by other systems described herein or other sensor systems not shown.

The process 300 can include Step 305, where a sensor activation command can be received by the sensor 120. For instance, the photodetector 228 or LC light detector 236 can receive via modulated light the sensor activation command, which can be processed by the processor 222. In Step 310, in response to receipt of the sensor activation command, tissue of a patient can be irradiated with one or more wavelengths of light by the light source 226. In Step 315, the photodetector 228 can detect the light after attenuation by the tissue of the patient and generate a detector signal responsive to the detected light. In Step 320, the photodetector 228 can output the detector signal. In optional Step 325, parameter measurements can be determined based on the detector signal by the processor 222. The determined parameter measurements can include one or more physiological measurements such as a blood glucose level, total hemoglobin, SpO2, methemoglobin, carboxyhemoglobin, pulse rate, perfusion, hydration, or pH, as examples. In Step 330, measurement or signal data can be encoded to transform the data from one format to another format for transmission. In an embodiment, the data can include the detector signal or one or more determined parameter measurements. In Step 335, the light source 226 or LC light source 234 can be activated to transmit the encoded data using modulated light. In optional Step 340, confirmation of data transfer can be received by the sensor 120. For instance, the photodetector 228 or LC light detector 236 can receive via modulated light the confirmation of data transfer, which can be processed by the processor 222. The confirmation of data transfer can indicate that the data transmitted by the light source 226 or LC light source 234 has been received by the monitor 110 without error.

FIG. 4 illustrates an instrument measurement process 400, according to an embodiment of the disclosure. The process 400 illustrates an example mode of operation of the monitor 110 in the patient monitoring system 100A of FIG. 1 and may be implemented by the various components of the monitor 110 of FIG. 2A. For convenience, the process 400 is described in the context of the patient monitoring system 100A of FIG. 1 and the monitor 110 of FIG. 2A, but the process 400 but may instead be implemented by other systems described herein or other monitor systems not shown.

The process 400 can include Step 405, where a sensor activation command can be transmitted or communicated by the monitor 110 to the sensor 120. For instance, the LC transmitter 210 can transmit the sensor activation command to the sensor 120 using modulated light. In Step 410, light, such as modulated light, that may be transmitted by the sensor 120 can be detected by the LC receiver 212 to generate a detected light signal. In Step 415, the detected light signal can be demodulated by the processor 202 to extract an information-bearing signal that includes the encoded data sent by the sensor 120. In Step 420, the demodulated signal can be decoded by the processor 202. In optional Step 425, parameter measurements can be determined based on the decoded signal by the processor 202. The determined parameter measurements can include one or more physiological measurements such as a blood glucose level, total hemoglobin, SpO2, methemoglobin, carboxyhemoglobin, pulse rate, perfusion, hydration, or pH, as examples. In Step 430, one or more of the parameter measurements can be displayed on the user interface 206. In optional Step 435, a confirmation of data transfer can be transmitted or communicated via modulated light to the sensor 120 to indicate that the received data has been received by the monitor 110 without error.

Although LC described in the processes 300 and 400 and in other parts of this disclosure may have been explained using the examples of communicating activation instructions, confirmation instructions, and measurement data via LC, the LC between the monitor 110 and the sensor 120 may not limited to such communications. Instead, the LC between the monitor 110 and the sensor 120 can be used to communicate any information between the monitor 110 and the sensor 120. For example, the LC between the monitor 110 and the sensor 120 can be used to communicate programming or setup data to manage the functionality of the monitor 110 or the sensor 120, security or authentication data to control access to the monitor 110 or the sensor 120 or associated measurement data, verification of device operation status to confirm the monitor 110 or the sensor 120 may be properly functioning for use in patient monitoring, or error troubleshooting for the monitor 110 or the sensor 120 to assist in addressing operation or functionality issues. Moreover, in some embodiments, the monitor 110 and the sensor 120 can share one or more common LC schemes or protocols to facilitate successful LC between the monitor 110 and the sensor 120. The one or more LC schemes or protocols, for instance, can define the modulation used to communicate information or how or when certain devices may engage in LC or use one or more other communication mechanisms for communication of information.

FIG. 5 illustrates LC between the monitor 110 and sensor 120 in a patient monitoring system 100B, according to an embodiment of the disclosure. In FIG. 5, the monitor 110 and sensor 120 are each illustrated as having an additional LC transmitter and receiver. The monitor 110 includes LC receiver 116 capable of light, photo, or video capture and LC transmitter 118 capable of transmitting light L₂, such as modulated light. The sensor 120 includes LC transmitter 126 capable of transmitting light L₃, such as modulated light, and LC receiver 128 capable of light, photo, or video capture. Advantageously, in certain embodiments, the LC transmitters 118, 126 and LC receivers 116, 128 can enable the monitor 110 and sensor 120 to transmit or detect light while performing other functions. For instance, the sensor 120 can simultaneously irradiate tissue of a patient using the emitters 122 and transmit information, such as sensor signals or readings, via light L₃ using the LC transmitter 126. Further, although the LC transmitters 118, 126 and LC receivers 116, 128 are shown as located on a front-top of the monitor 110 or sensor 120, the LC transmitters 118, 126 and LC receivers 116, 128 can be located in other positions, such as for example the back or bottom of the monitor 110 or sensor 120, in other implementations.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out all together (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

Moreover, all publications, patents, and patent applications mentioned in this specification are herein incorporated by reference to the same extent as if each individual publication, patent, or patent application was specifically and individually indicated to be incorporated by reference.

What is claimed is:

1. A physiological monitoring system configured to process signals responsive to light attenuated by body tissue carrying pulsing blood, the system configured to determine measurement values for one or more physiological conditions of a patient being monitored, the system comprising:
a noninvasive physiological sensor comprising a processor configured to:
in response to receiving a sensor activation command, communicate a first drive signal to a light source of said noninvasive physiological sensor to irradiate tissue of a patient with light,
receive a detector signal output by a detector of said noninvasive physiological sensor responsive to detected light after attenuation by said tissue carrying pulsing blood, and
communicate a second drive signal to said light source of said noninvasive physiological sensor to transmit a communication signal responsive to said detector signal output by said detector of said noninvasive physiological sensor; and
a patient monitor comprising a processor configured to:
communicate said sensor activation command to said noninvasive physiological sensor,
receive a detector signal output by a detector of said patient monitor responsive to detected light transmitted by said noninvasive physiological sensor, said detector signal output by said detector of said patient monitor responsive to said communication signal,
determine measurement values of one or more physiological parameters of said patient based at least on said detector signal output by said detector of said patient monitor, and
output said measurement values of said one or more physiological parameters for display on a display of said patient monitor.

2. The system of claim 1, wherein said measurement values of said one or more physiological parameters comprise measurement values for blood glucose, total hemoglobin, SpO2, methemoglobin, or carboxyhemoglobin.

3. A method of communicating optically between a noninvasive physiological sensor and a patient monitor, the method comprising:
irradiating tissue of a patient with light using a first light source of a sensor;
detecting with a detector of said sensor said light after attenuation by said tissue;
outputting from said detector of said sensor a detector signal responsive to said detected light; and
transmitting, with a second light source of the sensor, a transmission signal responsive to said detector signal, wherein said transmitting comprises emitting light.

4. The method of claim 3, wherein said transmitting comprises emitting light having an intensity that is modulated responsive to said detector signal.

5. The method of claim 3, wherein said transmitting comprises emitting light having a frequency that is modulated responsive to said detector signal.

6. The method of claim 3, wherein said first light source and said second light source comprise the same light source.

7. The method of claim 3, wherein said first light source and said second light source comprise different light sources.

8. The method of claim 3, wherein said irradiating is performed in response to receiving, at said sensor, a sensor activation command from a patient monitor.

9. The method of claim 3, further comprising determining one or more measurement values of physiological parameters of said patient based at least on said detector signal using a processor of said sensor.

10. The method of claim 9, wherein said measurement values of said one or more physiological parameters comprise measurement values for blood glucose, total hemoglobin, SpO2, methemoglobin, or carboxyhemoglobin.

11. The method of claim 3, further comprising:
receiving at a patient monitor said transmission signal detected using a detector of said patient monitor;
processing said detected transmission signal using a processor of said patient monitor to determine measurement values of one or more physiological parameters of said patient; and
displaying said measurement values on a display of said patient monitor.

12. The method of claim 11, further comprising communicating a confirmation of data transfer, using said patient monitor, upon receipt of said transmission signal without error.

13. The method of claim 11, further comprising demodulating said transmission signal and decoding said demodulated signal using said processor of said sensor.

14. A method of establishing an optical communication path between a sensor and a patient monitor, the method comprising:
activating a light source of a patient monitor to emit monitor communication light;
detecting said monitor communication light at a sensor configured to irradiate body tissue of a patient with light at different wavelengths, detect said light after attenuation by said body tissue carrying pulsing blood, and output a detector signal responsive to said detected light;
activating a light source of said sensor to emit sensor communication light; and
detecting said sensor communication light at said patient monitor.

15. The method of claim 14, further comprising encoding said detector signal into said sensor communication light using a processor of said sensor.

16. The method of claim 15, further comprising processing said detector signal using a processor of said patient monitor to determine measurement values of one or more physiological parameters of said patient.

17. The method of claim 14, further comprising encoding a sensor activation command into said monitor communication light using a processor of said patient monitor, said sensor activation command causing said sensor to irradiate said body tissue.

18. The method of claim 14, further comprising encoding a data receipt acknowledgement into said sensor communication light using a processor of said sensor.

19. The method of claim 14, further comprising encoding a data receipt acknowledgement into said monitor communication light using a processor of said patient monitor.
